(19) United States
(12) Patent Application Publication
(10) Pub. No.: US 2008/0175903 A1
(43) Pub. Date: Jul. 24, 2008

(54) Treatment of anxiety with eszopiclone

(75) Inventors: Seth C. Hopkins, Clinton, MA (US); Mark A. Varney, Laguna Niguel, CA (US); Tushar Misra, Leesburg, VA (US); Gary Maier, Sterling, MA (US); Judy Caron, Westwood, MA (US); Randall S. Wagner, Boylston, MA (US)

Correspondence Address: Peter F. Corless, P.O. Box 55874, Boston, MA, 02205, US

(73) Assignee: Sepracor Inc., Marlborough, MA (US)

(21) Appl. No.: 11/998,839

(22) Filed: Dec. 1, 2007

Related U.S. Application Data
(60) Provisional application No. 60/868,279, filed on Dec. 1, 2006.

(51) Int. Cl. A61K 31/4985; A61K 9/54; A61P 25/22

(52) U.S. Cl. 424/458; 514/249

(57) ABSTRACT

The present disclosure provides a unit dosage form with an anxiolytic dosage of zopiclone particularly eszopiclone. Also provided is a method for treatment or prophylaxis of anxiety using a subsedative dosage of zopiclone particularly eszopiclone.

The present application claims the benefit of U.S. provisional application No. 60/868,279 filed Dec. 1, 2006, which is incorporated herein by reference in its entirety.

### BACKGROUND

Anxiety neurosis is experienced primarily as a combination of indistinct fears and physical symptoms, for example, cardiopalmus, palmospasm of fingers, thirst, perspiration, frequent urination and respiratory distress. Panic disorder is recognized as a category of anxiety neurosis, characterized by repeated panic attacks and other symptoms, e.g., respiratory distress, palpitation, perspiration, choking feeling and dysaethesia, together with fear of death or insanity.

Treatment of anxiety neurosis, including panic disorder, relies primarily on the use of selective serotonin reuptake inhibitors (SSRIs) or benzodiazepine anxiolytic drugs. However, SSRI use can induce significant sexual side effects. Benzodiazepine use can induce side effects of hypersedation, break-off phenomenon and addiction. The discovery of new anxiolytic agents thus would be desirable.

Eszopiclone is a cyclopyrrolone that has the chemical name (+)-(5S)-6-(chloropyridin-2-yl)-7-oxo-6,7-dihydro-5H-pyrrolo[3,4-b]pyrazin-5-yl 4-methyl-piperazine-1-carboxylate.

Eszopiclone is the S-(+)-optical isomer of the compound zopiclone, which is described in U.S. Pat. Nos. 6,319,926 and 6,444,673, and in Goa and Heel (Drugs, 32:48-65 (1986)) and in U.S. Pat. Nos. 3,862,149 and 4,220,646. This isomer, which will hereinafter be referred to by its USAN-approved generic name, eszopiclone, includes the optically pure and the substantially optically pure (e.g., 90%, 95% or 99% optical purity) S-(+)-zopiclone isomer.

Zopiclone was the first of a chemically distinct class of hypnotic compounds—cyclopyrrolones—with a psychotherapeutic profile of efficacy similar to the benzodiazepines. Zopiclone, however, causes less residual sedation and less slowing of reaction times than the benzodiazepines, and it offers an improved therapeutic index over benzodiazepines.

### SUMMARY

It has now been discovered that selected dosages and formulations of eszopiclone are anxiolytic. Such anxiolytic effects of eszopiclone have been assessed in both primate and human subjects.

In a preferred aspect, the invention includes modified administration of eszopiclone to treatment of anxiety. Preferably, eszopiclone is administered as a sustained release formulation.

It also has been discovered that administration of low dosages of eszopiclone to a subject at times other than at the subject's bedtime, exert an unexpected anxiolytic activity.

In a disclosed embodiment, the dosage can be sufficiently low that the eszopiclone remains at a subsedative plasma concentration during the entire transit of the eszopiclone through the subject's body.

In a further embodiment, the dosage form can be sufficiently low that the eszopiclone remains at a subsedative plasma concentration during at least a substantial portion of the transit of the eszopiclone through the subject's body. For example, in preferred aspects, eszopiclone may be administered at dosage level where the Cₘₐₓ is about the same or even exceeds the Cₘₐₓ of a reference instantaneous release one (1) mg eszopiclone formulation (“reference eszopiclone formulation”), but where the formulation provides a greater AUC (area under the plasma concentration time curve of eszopiclone) than a reference eszopiclone formulation, e.g. where the administered eszopiclone formulation provides an AUC that is at least 110, 120, 130, 140, 150, 160, 170, 180, 190, 200, 210, 220, 230, 240, 250, 260, 270, 280, 290 or 300 percent greater than a reference eszopiclone formulation. An AUC of from about 130 to about 400 percent greater, or from about 150 to about 300 or 350 percent greater than the AUC of a reference eszopiclone formulation will be suitable for many applications. In some aspects, as will be specifically indicated herein, the AUC values of a present unit dosage form relative to an instantaneous release reference eszopiclone formulation are indicated on the basis of where each of the present unit dosage form and the instantaneous release reference eszopiclone formulation contains the same amount by weight of eszopiclone (rather than with respect to an instantaneous release formulation that contains one (1) mg of eszopiclone).

Additionally, in certain preferred aspects, the Cₘₐₓ of a present eszopiclone formulation will be no more than four times greater than the Cₘₐₓ of a reference eszopiclone formulation, more preferably no more than two or three times greater than the Cₘₐₓ of a reference eszopiclone formulation. In some aspects, it will be preferred that the Cₘₐₓ of a present eszopiclone formulation will be from 40 to 200 or 300 percent of the Cₘₐₓ of a reference eszopiclone formulation, such as up to 40, 50, 60, 70, 80, 90, 100, 110, 120, 130, 140, 150, 160, 170, 180, 190, 200, 220, 240, 260, 280 or 300 percent of the Cₘₐₓ of a reference eszopiclone formulation. In some aspects, as will be specifically indicated herein, the Cₘₐₓ values of a present unit dosage form relative to an instantaneous release reference eszopiclone formulation are indicated on the basis of where each of the present unit dosage form and the instantaneous release reference eszopiclone formulation contains the same amount by weight of eszopiclone (rather than with respect to an instantaneous release formulation that contains one (1) mg of eszopiclone).

In additional certain preferred aspects, the Tₘₐₓ of a present eszopiclone formulation will be no more than three or four times greater than the Tₘₐₓ of a reference eszopiclone formulation, more preferably no more than two the Tₘₐₓ of a reference eszopiclone formulation. In some aspects, it will be preferred that the Tₘₐₓ of a present eszopiclone formulation will be from 30 to 180 percent of the Tₘₐₓ of a reference eszopiclone formulation, such as up to 30, 40, 50, 60, 70, 80, 90, 100, 110, 120, 130, 140, 150, 160, 170, or 180 percent of the Tₘₐₓ of a reference eszopiclone formulation. In some aspects, as will be specifically indicated herein, the Tₘₐₓ values of a present unit dosage form relative to an instantaneous release reference eszopiclone formulation are indicated on the basis of where each of the present unit dosage form and the instantaneous release reference eszopiclone formulation contains the same amount by weight of eszopiclone (rather than with respect to an instantaneous release formulation that contains one (1) mg of eszopiclone).

In assessing AUC, Cₘₐₓ and Tₘₐₓ values of a present eszopiclone formulation relative to a reference eszopiclone formulation, preferably the assessment is made with respect to values derived where subjects to whom the present eszopiclone formulation and the reference eszopiclone formulation is administered each has similar characteristics, particularly where each subject has a similar weight (e.g. each subject has the same body weight or each subject is within 10 percent of the same body weight such as 70 kg) and each subject is in a fed condition or fasted condition. For such assessments, a fasted condition refers to a subject who has not eaten any food for at least 8 hours before administration of the eszopiclone formulation and does not eat any food for three hours following administration of the eszopiclone formulation. For such assessments, a fed condition refers to a subject who has eaten an entire meal at least one hour before administration of the eszopiclone formulation.

In many preferred aspects, a present eszopiclone formulation will contain less than 1 mg of eszopiclone. In other preferred aspects, however, as present eszopiclone formulation may contain greater amounts of eszopiclone, such as up to 5 mg/70 kg, drug/patient weight, 4 mg/70 kg, drug/patient weight, 3.5 mg/70 kg, drug/patient weight, 3 mg/70 kg, drug/patient weight, 2.5 mg/70 kg, drug/patient weight, 2 mg/70 kg, drug/patient weight, 1.5 mg/70 kg, drug/patient weight, or 1 mg/70 kg, drug/patient weight, preferably, however, where the formulation provides a subsedative plasma concentration during at least a substantial portion of the transit of the eszopiclone through the subject's body, as discussed above.

A subsedative plasma concentration of eszopiclone in a particular subject may vary with a number of factors, including the subject's circadian sleep cycle, time of dosage (e.g. whether the eszopiclone is administered within 2 to 4 hours of a subject's sleep cycle, or greater than 2 to 4 hours outside the subject's sleep cycle), subject's weight, subject's medical or psychiatric status, and other factors. For example, a relatively greater eszopiclone plasma concentration will be subsedative at periods outside of a subject's sleep cycle, e.g. from early morning to late afternoon (e.g. from 5:00 am to 6:00 pm) for a subject that has an evening sleep cycle. Some subjects may have distinct sleep cycles, e.g. a night-time worker may have a daytime sleep cycle and for such subjects a relatively greater eszopiclone plasma concentration will be subsedative at evening time periods (e.g. from 6:00 pm to 8:00 am). In any event, in many aspects, an eszopiclone plasma concentration of less than 25 ng/ml, 20 ng/ml, 15 ng/ml, 10 ng/ml, 5 ng/ml, 4 ng/ml, 3 ng/ml, 2 ng/ml, 1 ng/ml, such as 0.9 ng/ml, 0.8 ng/ml, 0.7 ng/ml, 0.6 ng/ml, 0.5 ng/ml, 0.4 ng/ml, 0.3 ng/ml 0.2 ng/ml or 0.1 ng/ml will be considered subsedative.

The present disclosure provides a method for providing anxiolysis or preventing anxiety using zopiclone, particularly (S)-zopiclone, at lower doses than those used to induce sedation, e.g. treating insomnia, or which are available through ingestion of commercially available formulations of this agent. Moreover, eszopiclone has improved safety, tolerability, and withdrawal liabilities compared to classical benzodiazepines used to treat anxiety.

Currently, eszopiclone is used principally as a sleep aid in dosages that provide at least moderate sedation. Moreover, when eszopiclone is used to treat or ameliorate a disease or condition other than insomnia (e.g., schizophrenia, convulsions, etc.), the dosages administered are sufficient to induce at least moderate sedation in the subject to whom eszopiclone is administered. Thus, the utility of eszopiclone within a range of subsedative dosages for treatment and prophylaxis of anxiety is unexpected, i.e. that a subsedative dose of eszopiclone would provide any beneficial result to a subject to whom such a dose was administered.

Prior to the present disclosure there was no recognition that dosages within a controlled subsedative dosage range exerted a clinically determinable anxiolytic effect.

Accordingly, one aspect of the present disclosure provides methods of inducing anxiolysis in a subject, such as a human subject. The methods can include administering to a subject in need of treatment for or prophylaxis of anxiety, a unit dosage form comprising an amount of or a sustained release component containing a compound according to Formula I:

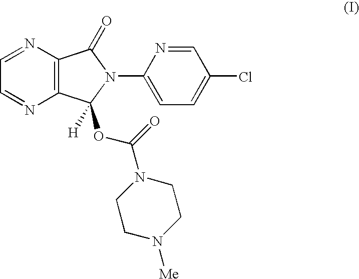

sufficient to induce anxiolysis and insufficient to induce moderate sedation in the subject. Various alternative embodiments can include providing a plasma concentration of the compound of Formula I sufficient to induce anxiolysis in a subject for at least about 6 hours while ensuring that the maximum plasma concentration (Cₘₐₓ) is insufficient to moderately sedate the subject for greater than 1 hour.

Exemplary types of anxiety treatable according to the instant method include panic attack, agoraphobia, acute stress disorder, specific phobia, panic disorder, psychoactive substance anxiety disorder, organic anxiety disorder, obsessive-compulsive anxiety disorder, posttraumatic stress disorder and generalized anxiety disorder.

In a further aspect, the disclosure provides unit dosage forms that preferably supply a subsedative dose of eszopiclone or a pharmaceutically acceptable salt, solvate, hydrate, enantiomer, racemate, polymorph, clathrate metabolite or prodrug thereof. In the disclosed unit dosage forms, the compound according to Formula I can be present in an amount or in a modified release form (e.g., sustained release component) effective to achieve a maximum plasma concentration (Cₘₐₓ) sufficient to induce anxiolysis, but insufficient to induce moderate sedation, in the subject to whom the unit dosage form is administered. The unit dosage form can include an amount of eszopiclone or a sustained release component sufficient to ameliorate or provide prophylaxis of anxiety in a subject. Various embodiments of the present unit dosage forms can induce anxiolysis in a subject for a period of at least about 6 hours without moderately sedating the subject. The unit dosage forms can be of use in the presently disclosed methods.

In a further aspect, the invention provides a use of zopiclone, particularly eszopiclone, for the treatment or prevention of anxiety.

In a yet further aspect, the invention provides a use of zopiclone, particularly eszopiclone, for the preparation of medicament for the treatment or prevention of anxiety.

Preferred methods and uses of the invention include identifying and/or selecting a subject, particularly a human subject, that is susceptible to or suffering from anxiety and thereafter administering to the identified and selected subject zopiclone, particularly eszopiclone. Such identification and selection may be made e.g. by a physician or other health professional.

Other objects and advantages of the disclosed subject matter will be apparent to those of skill in the art from the detailed description that follows.

### BRIEF DESCRIPTION OF THE DRAWINGS

FIG. 1 is a plot of mean (S)-zopiclone plasma concentration-time profiles following oral administration of (S)-zopiclone. Following dosing, the maximum mean (S)-zopiclone plasma concentrations were achieved at 0.5-1.5 hours post-dose under fasted conditions and at 2-3 hours post-dose under fed conditions, followed by a monoexponential decline in concentrations.

FIG. 2 is a plot showing the concentration-dependent potentiation of GABA evoked currents by (S)-zopiclone. For all experiments, human cDNA encoding each subunit was injected in oocytes in a stoichiometry of 1:1:3 (alpha, beta, gamma subunits). Approximately 24 hours later, GABA evoked currents (using a concentration of GABA equal to the EC₁₀) were recorded from oocytes using a two-electrode voltage clamp on a robotic oocyte platform and a constant voltage of −80 mV. GABA was applied to the oocytes via a perfusion system for 20 seconds and then removed. An inward deflection of the current trace was recorded and subsequently compared to the responses resulting from applying the same concentration of GABA plus varying concentrations of the test compound ((S)-zopiclone). A doubling of the GABA evoked current is represented as a potentiation ratio of 1.0.

FIG. 3 is a plot of rates of non-suppressed and suppressed responding by rhesus monkeys trained in a conflict procedure. Monkeys were injected i.v. with cumulative doses of (S)-zopiclone. Data are mean±S.E.M. for N=4 monkeys. Points above “V” represent data following drug vehicle administration. Note that *P<0.05 vs. V (control). Experimental methodology was identical to that described by Rowlett J K et al. (2006).

FIG. 4 shows EEG plot results of Example 4 which follows.

### DETAILED DESCRIPTION

The present invention includes novel formulations and unit dosage forms of zopiclone particularly eszopiclone and methods, including methods of using these formulations and dosage forms, for the treatment or prophylaxis of anxiety.

We have now demonstrated in human subjects anxiolytic effects of eszopiclone, including with separation from sedative effects. In particular, EEG studies have shown anxiolytic effects in the absence of sedative effects at varying doses of eszopiclone in adult human subjects.

In preferred aspects, administration of present eszopiclone formulations will provide an increase (e.g. at least 10, 20, 30, 40, 50, 60, 70, 80, 90 or 100 percent) in beta EEG activity relative to a placebo control. More preferably, administration of present eszopiclone formulations will provide an increase (e.g. at least 10, 20, 30, 40, 50, 60, 70, 80, 90 or 100 percent) in beta EEG activity relative to a placebo control, but with minimal (e.g. less 50, 40, 40, 20, or 10 percent) increase in delta EEG activity relative to a placebo control.

Eszopiclone is a non-benzodiazepine hypnotic agent that is a pyrrolopyrazine derivative of the cyclopyrrolone class. The chemical name of eszopiclone is (+)-(5S)-6-(chloropyridin-2-yl)-7-oxo-6,7-dihydro-5H-pyrrolo[3,4-b]pyrazin-5-yl 4-methyl-piperazine-1-carboxylate. Its molecular weight is 388.81, and its empirical formula is C₁₇H₁₇ClN₆O₃. Racemic zopiclone and some of its uses are described by U.S. Pat. Nos. 3,862,149 and 4,220,646. Uses of the optically pure (+) and (−) enantiomers of the drug (i.e., (+)-zopiclone and (−)-zopiclone) are described by U.S. Pat. No. 5,786,357 and WO 93/10788, respectively.

The present disclosure emerges from the recognition that a controlled range of dosages of eszopiclone, not previously disclosed in the art, exerts an unexpected and clinically determinable anxiolytic effect.

In preferred aspects, a modified administration of eszopiclone (e.g. a nonimmediate release formulation) is utilized in a relatively low dose formulation (e.g. a nonimmediate release formulation containing up to about 4.0 mg, 3.5 mg, 3.0 mg, 2.5 mg, 2.0 mg, 1.5 mg, 1.25 mg, 1.0 mg, 0.9 mg or less of eszopiclone) to provide an anxiolytic effect, preferably without undesired sedative effects. Sustained release formulations are preferred, such as an oral sustained release formulation (e.g. tablet, capsule) that may be administered one or more times per day. Unit dosages containing from 0.4 mg to 1.5 mg of eszopiclone or from 0.5 mg to 0.9 mg of eszopiclone, such as 0.4, 0.5, 0.6, 0.7, 0.8, 0.9, 1.0, 1.1, 1.2, 1.3, 1.4 or 1.5 mg of eszopiclone, may be preferred for many applications.

As discussed, it also may be preferred to administer a nonimmediate release formulation at times other than a subject's typical bedtime, e.g. other than from 8:00 pm to 4:00 am, more preferably at least two or four hours outside or beyond such times. Administration in the morning (e.g. from 6:00 am to noon) or afternoon (e.g. noon to 5:00 pm) may be preferred to enhance anxiolytic effects, in the significant or complete absence of undesired sedative effects.

As an alternative to, or in conjunction, with a modified administration of eszopiclone, one or more immediate release formulations of eszopiclone may be administered to a subject, preferably other than at a subject's bedtime. Such immediate release formulations contain low dosages of eszopiclone, e.g. less than 1.6 mg, 1.5 mg, 1.4 mg, 1.3 mg, 1.2 mg, 1.1 mg, 1.0 mg, 0.9 mg, 0.8 mg, 0.7 mg, 0.6 mg, 0.5 mg or 0.4 mg of eszopiclone. Preferably, such immediate release formulations are administered other than a subject's bedtime, e.g. other than from 8:00 pm to 4:00 am. Immediate release formulations preferably would be administered to a subject multiple times during a 24-hour period, e.g. 2, 3 or 4 or more times during a 24 hour period.

### Definitions

As used herein, and as would be understood by the person of skill in the art, to which the disclosure pertains, the recitation of the terms “eszopiclone” and “S-(+)-zopiclone” include pharmaceutically acceptable salts, hydrates, solvates, clathrates, and polymorphs of S-(+)-zopiclone. As used herein, the recitation of the terms “eszopiclone” and “S-(+)-zopiclone” refers to eszopiclone having an enantiomeric excess (e.e.) greater than 90%.

The term “enantiomeric excess” is related to the older term “optical purity” in that both are measures of the same phenomenon. The value of e.e. will be a number from 0 to 100, zero being racemic and 100 being a pure, single enantiomer. In the case of eszopiclone, e.e. of greater than 95% is preferred; e.e. of greater than 98% is more preferred; and e.e. of greater than 99% is most preferred.

As used herein, and unless otherwise indicated, a composition that is “essentially free” of a compound means that the composition contains less than about 20% by weight, such as less than about 10% by weight, less than about 5% by weight, or less than about 3% by weight of the compound, still more preferably less than about 1% by weight or less than 0.1% by weight.

As used herein, and unless otherwise indicated, the term “enantiomerically pure” means a stereomerically pure composition of a compound having one chiral center. An equivalent term used herein when referring to a composition comprising eszopiclone is “essentially free of antipodal enantiomer of said compound”

The term “antianxiety dose”, as used herein, refers to an amount of eszopiclone necessary to prevent anxiety in a human susceptible to this condition, or to treat anxiety in a subject suffering from anxiety. An antianxiety dose of eszopiclone is preferably a subsedative dose, i.e., a dose prevents or treats anxiety, but preferably does not induce moderate sedation in the subject to whom the antianxiety dose is administered.

As used herein the term “anxiety” refers to an anxiety disorder. Examples of anxiety disorders treatable by the compositions and methods disclosed herein include, but are not limited to: panic attack, agoraphobia, acute stress disorder, specific phobia, panic disorder, psychoactive substance anxiety disorder, organic anxiety disorder, obsessive-compulsive anxiety disorder, posttraumatic stress disorder and generalized anxiety disorder. Anxiety as referred to herein also includes situational anxiety (e.g. as experienced by a performer prior to a performance).

The named anxiety disorders have been characterized in the DSM-IV-R. Diagnostic and Statistical Manual of Mental Disorders, Revised, 4th Ed. (1994). The DSM-IV-R was prepared by the Task Force on Nomenclature and Statistics of the American Psychiatric Association, and provides clear descriptions of diagnostic categories.

The terms “effective amount,” “therapeutically effective amount,” or “pharmaceutically effective amount” of eszopiclone in unit dosage form of the composition depends upon a number of factors. Included among these factors is the quantity of the other ingredients when used. An effective amount of eszopiclone ranges from about 0.1% to about 100% by weight based on the total weight of the composition but, in any event, is sufficient to observe the anticipated benefit.

As used herein, the term “extended release” or “sustained release” refers to a drug formulation that provides for more gradual release of a drug over an extended period of time relative to an immediate release formulation of the drug.

As used herein, the term “modified” refers to a drug containing formulation in which release is not immediate. That is, in a modified formulation, administration of a formulation does not result in immediate release of the drug or active agent into an absorption pool. The term “modified” is used synonymously with the term “nonimmediate release” as defined in Remington, The Science and Practice of Pharmacy, 19^{th} ed. (Easton, Pa., Mack Publishing Company 1995). The term “modified release” as used herein includes extended release, sustained release, delayed release, pulsatile release and controlled release formulations.

As used herein, the term “Cₘₐₓ” refers to the maximum observed plasma concentration of drug (in particular, eszopiclone).

As used herein, the term “Tₘₐₓ” refers to the time to the maximum observed plasma concentration of drug (in particular, eszopiclone).

As used herein, the term “AUC” means the area under the plasma concentration time curve of the active agent (in particular, eszopiclone) as measured using the trapezoidal rule.

As used herein, unless specifically indicated otherwise herein, the term “reference eszopiclone formulation” refers to an instantaneous release formulation that contains one (1) mg eszopiclone and where such 1 mg eszopiclone formulation provides in a 70 kg subject a Cₘₐₓ of eszopiclone of 10 ng/ml, a Tₘₐₓ of 1.5 hours and a drug half-life of 6 hours. Additionally, where specifically indicated to be the case herein, a “reference eszopiclone formulation” refers to an instantaneous release formulation that has the same amount of eszopiclone by weight as the specified eszopiclone formulation (e.g. unit dosage formulation) of the present invention. In all cases, the term “reference eszopiclone formulation” specifies an instantaneous release eszopiclone formulation.

As used herein, and unless otherwise specified, the terms “treat,” “treating” and “treatment” refer to the eradication or amelioration of anxiety or one or more symptoms associated with the disease. In certain embodiments, the terms refer to minimizing the intensification of anxiety or a symptom thereof resulting from the administration of one or more prophylactic or therapeutic agents to a subject with such a disease.

As used herein, and unless otherwise specified, the terms “prevent,” “preventing” and “prevention” refer to the prevention of the onset, recurrence or intensification of anxiety or a symptom thereof. The terms “prevent,” “preventing” and “prevention” include ameliorating and/or reducing the occurrence of symptoms of anxiety.

As used herein, the phrases “induce anxiolysis” and “inducing anxiolysis” mean treating, preventing or otherwise reducing the severity of at least one symptom associated with anxiety or an anxiety disorder, including acute anxiety, chronic anxiety, general anxiety disorder caused by psychologic and/or physiologic factors, and other anxiety disorders such as panic disorders, mood anxiety, panic attacks, phobias, obsessive-compulsive disorders, or post traumatic distress disorder. Symptoms associated with acute anxiety include, but are not limited to, a fear of losing control of one's own actions, a sense of terror arising from no apparent reason, and a dread of catastrophe. Symptoms associated with chronic anxiety include, but are not limited to, uneasiness, nervousness, nagging uncertainty about future events, headache, fatigue, and subacute autonomic symptoms.

“Clinically determinable” refers to a quality and/or quantity of a state, disease or condition or change in a state, condition or disease that is detectable by tests and parameters recognized in the relevant art as diagnostic of the state, condition or disease and its presence, progression, stasis or reversal.

“Conflict Procedure,” as used herein refers to the rhesus monkey conflict procedure reported by Rowlett et al. (Psychopharmacology (2006) 184:201-211). Antianxiety dosage determination in rhesus monkeys using the procedure set forth by Rowlett et al. is correlative with antianxiety dosages in humans. The conflict procedure is a method of determining the parameters correlating a clinically determinable effect of eszopiclone in rhesus monkeys with analogous effects in a human subject. The results of conflict procedures in rhesus monkeys are closely correlated with dosages of agents providing anxiolysis in humans. Accordingly, the conflict procedure is a method of determining whether a dosage of eszopiclone provides clinically determinable treatment or prophylaxis of anxiety in humans.

“Subsedative” refers to both the compositions (e.g., unit dosage form) and dosages of use in the methods disclosed herein. Regarding compositions, subsedative can refer to a composition that when administered to a subject weighing 70 kg, would not produce clinically determinable moderate sedation as this term defined by the American Society of Anesthesiologists. Preferred subsedative dosages are those that substantially correlate to anxiolytic and subsedative dosages in the Rowlett rhesus monkey model.

“Moderate Sedation” sedation refers to the state in which the subject's plasma concentration maximum (Cₘₐₓ) of eszopiclone becomes as high as that achievable by administration to a subject of commercially available forms of eszopiclone, or within ranges higher than these ranges, e.g. a Cₘₐₓ of 30 ng/ml or 40 ng/ml or greater. This term “moderate sedation” also may refer to a condition induced in a subject by a plasma concentration maximum (Cₘₐₓ) of eszopiclone at least sufficient to meet the guidelines of the American Society of Anesthesiologists (ASA) for Moderate Sedation, or other known performance-based test.

The term “commercially available forms of eszopiclone” or similar phrase refers to a commercially available immediate release oral dosage formulation that contains 1 mg, 2 mg or 3 mg of eszopiclone, which are typically administered as a sleep aid one time per 24 hour period at subject's bedtime (e.g. administering between 6:00 pm and 12:00 pm).

The term “pharmaceutically acceptable salt” refers to salts prepared from pharmaceutically acceptable, non-toxic acids or bases including inorganic acids and bases and organic acids and bases. Suitable pharmaceutically acceptable acid addition salts for eszopiclone include acetic, benzenesulfonic(besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like.

The term “solvate” refers to a compound—in this case eszopiclone—in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The term “prodrug” is accorded a meaning herein such that prodrugs of eszopiclone derivatives do not encompass zopiclone, zopiclone-N-oxide, or N-desmethylzopiclone.

As used herein, and unless otherwise indicated, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, amino acids, hydroxyalkylamines, heterocyclic and heteroaromatic amines, and polyether amines.

As used herein, and unless otherwise indicated, the term “biohydrolyzable ester” means an ester of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, alkoxyacyloxy esters, alkyl acylamino alkyl esters, and choline esters.

As used herein, and unless otherwise indicated, the term “biohydrolyzable amide” means an amide of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable amides include, but are not limited to, lower alkyl amides, α-amino acid amides, alkoxyacyl amides, and alkylaminoalkylcarbonyl amides.

### Compositions

In one aspect, the present disclosure emerges from the recognition that a controlled range of dosages of eszopiclone, when administered to a subject in need thereof, exerts an unexpected and clinically determinable anxiolytic effect.

Accordingly, there is provided a unit dosage form supplying a dose, such as a subsedative dose, of eszopiclone (Formula I) or a pharmaceutically acceptable salt, solvate, hydrate, polymorph, clathrate, or prodrug thereof:

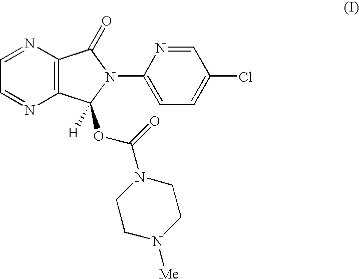

In the unit dosage forms of the present disclosure, the compound eszopiclone can be present in an amount or in a sustained release component effective to achieve a maximum plasma concentration (Cₘₐₓ) sufficient to induce anxiolysis in the subject to whom the unit dosage form is administered. The unit dosage form can include an amount of eszopiclone sufficient to ameliorate, treat or provide prophylaxis of anxiety in a subject.

In certain embodiments, the dosage and Cₘₐₓ are preferably sub-sedative (e.g., not inducing moderate sedation) through the duration of its transit through the subject to which it is administered.

There are numerous ways to describe various unit dosage forms of the present disclosure. In an exemplary embodiment, in which an otherwise healthy adult human is treated for anxiety, a unit dosage form of the present disclosure can provide an anxiolytic dose of eszopiclone reaching a maximum plasma concentration (Cₘₐₓ) in the subject of from about 0.1 ng/mL to about 25 ng/mL, such as from about 0.5 ng/mL to about 20 ng/mL, or from about 1 ng/mL to about 10 ng/mL, such as from about 2 ng/mL to about 8 ng/mL, or from about 3 ng/mL to about 5 ng/mL of eszopiclone.

A general unit dosage form can include eszopiclone in an amount sufficient to achieve a maximum plasma concentration (Cₘₐₓ) of not more than about 20 ng/mL of said compound, such as not more than about 8 ng/mL of said compound.

In exemplary embodiments, the unit dosage form can include eszopiclone in an amount sufficient to provide a dose of less than about 4 mg/70 kg, drug/patient weight, such as from about 0.25 mg/70 kg to about 0.9 mg/70 kg, drug/patient weight, or from about 0.5 mg/70 kg to about 0.9 mg/70 kg, drug/patient. In another exemplary embodiment, the unit dosage form can include eszopiclone in an amount less than about 1 mg/70 kg, drug/patient weight.

In additional exemplary embodiments, a modified release (e.g. sustained release) unit dosage form can include eszopiclone in an amount sufficient to provide a dose of less than about 4 mg/70 kg, drug/patient weight, such as from about 0.25 mg/70 kg to about 0.9 mg/70 kg, 1.0 mg/70 kg, 1.25 mg/70 kg, 1.5 mg/70 kg, 1.75 mg/70 kg or 2.0 mg/70 kg drug/patient weight. In another exemplary embodiment, the unit dosage form can include eszopiclone in an amount less than about 2.0 mg/70 kg, drug/patient weight or 1.5 mg/70 kg, drug/patient weight.

A unit dosage form intended for a healthy 70 kg adult subject that includes from about 0.4 mg to about 0.9 mg, 1.0 mg, 1.25, 1.5 mg, 2.0 mg, 2.5 mg, 3.0 mg or 3.5 mg of eszopiclone may be preferred. In certain aspects, the “healthy” subject does not require treatment for any one or more of the following conditions, insomnia, epilepsy, muscle spasms, stuttering, schizophrenia, or chronic pain. In other certain aspects, the subject does not require treatment for any condition other than anxiety, which could be treated by the dosages of eszopiclone set forth herein. Equivalent, proportional dosages (less or more eszopiclone, respectively) for lighter and heavier subjects as well as subjects suffering from conditions in which a lower or higher dosage is implicated are included within this invention.

An exemplary dosage form includes an amount of eszopiclone that correlates in a human subject to an anxiolytic dosage determined by the rhesus monkey conflict procedure reported by Rowlett et al. (Psychopharmacology (2006) 184:201-211).

Racemic zopiclone is commercially available and can be made using various methods, such as those disclosed in U.S. Pat. Nos. 3,862,149 and 4,220,646. Eszopiclone is also commercially available or it may be prepared from racemic zopiclone using standard methods, such as chiral-phase chromatography, resolution of an optically active salt, stereoselective enzymatic catalysis by means of an appropriate microorganism, or asymmetric synthesis. U.S. Pat. No. 6,319,926 discloses methods for making (+) zopiclone, including resolution from racemic zopiclone by means of an optically active acid, such as D(+)-O,O′-dibenzoyltartaric acid.

The amount of eszopiclone in the unit dosage forms of the present disclosure can vary depending on the formulation selected, the age, weight and general health of the subject to whom the formulation is to be administered. For example, it is generally preferred to provide pediatric and geriatric subjects, as well as subjects with impaired renal or hepatic function lower dosages of eszopiclone than might be provided to healthy adult subjects. As those skilled in the art recognize, many factors that modify the action of the anxiolytic composition and second active agents herein will be taken into account by the treating physician including, but not limited to, such factors as age, body weight, sex, diet and condition of the patient, time of administration, rate and route of administration, psychiatric condition, other diseases, and so forth. Titration of dosage for individual patients, up to the maximal dose permits attainment of functionally effective doses. Optimal dosages for a given set of conditions can be ascertained by those skilled in the art using conventional dosage determination tests in view of experimental data provided herein. Thus, unit dosage forms for particular patient classes are envisioned, in which the amount of active agent in the unit dosage form is varied depending on the patient class.

In an embodiment, the unit dosage form can include 0.1% by weight to 1% by weight or more, such as 2% by weight or more, or 3% or more by weight of eszopiclone.

As both (+)- and (−)-zopiclone are routinely administered to human subjects, the unit dosage formulations of the instant disclosure can include a range of the (+)- and (−)-zopiclone enantiomers.

In other exemplary embodiments, eszopiclone is present in said unit dosage form is present in at least about 99.5% enantiomeric excess, such as at least about 99.9% enantiomeric excess. In a further embodiment, the unit dosage form is essentially free of an antipodal enantiomer of eszopiclone.

Formulations and treatment methods of the invention will be useful for treatment of a variety of subjects, particularly a variety of mammals especially humans. Other mammals also may benefit from the treatment methods and formulations including horses and livestock as well as pets e.g. dogs and cats.

In addition to zopiclone particularly eszopiclone, pharmaceutical compositions and unit dosage forms of the present disclosure typically also include one or more pharmaceutically acceptable excipients or diluents.

Single unit dosage forms of the present disclosure can be suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets including orally dissolving tablets; thin films; caplets; granules, capsules, such as soft elastic gelatin capsules; cachets; troches; lozenges; dispersions; suppositories; ointments; cataplasms (poultices); pastes; powders; dressings; creams; plasters; solutions; patches; liquid sprays; metered and unmetered aerosols (e.g., nasal sprays or inhalers); drops; lyophilized compositions; transdermal patches; gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; sterile solids (e.g., crystalline or amorphous solids) that can be reconstituted to provide liquid dosage forms suitable for parenteral administration to a patient; and as components of autoinjector devices.

The composition, shape, and type of dosage forms of the present disclosure will typically vary depending on their use. For example, a dosage form used in the acute treatment of a disorder may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water.

Pharmaceutical compositions of the present disclosure may be administered by any suitable route of administration that provides a patient with a therapeutically effective dosage of eszopiclone. Typically, the eszopiclone pharmaceutical compositions described herein will be formulated for oral administration or for inhalation. Suitable dosage forms include tablets, troches, cachets, caplets, capsules, including hard and soft gelatin capsules, and the like. Tablet forms, however, remain a particularly useful dosage form because of advantages afforded both the patient (e.g., accuracy of dosage, compactness, portability, blandness of taste and ease of administration) and to the manufacturer (e.g., simplicity and economy of preparation, stability and convenience in packaging, shipping and dispensing).

The pharmaceutical compositions may further include a “pharmaceutically acceptable carrier”. This expression includes one or more inert excipients, including starches, polyols, granulating agents, microcrystalline cellulose, diluents, lubricants, binders, disintegrating agents, and the like. Depending on the particular route of administration, a variety of pharmaceutically acceptable carriers well known in the art may be used, including solid or liquid fillers, diluents, hydrotropies, surface-active agents, and encapsulating substances. “Pharmaceutically acceptable carrier” also encompasses sustained release means. Compositions of the present disclosure also optionally include other therapeutic ingredients, anti-caking agents, preservatives, sweetening agents, colorants, flavors, desiccants, plasticizers, dyes, and the like. Any such optional ingredient is compatible with eszopiclone to insure the stability of the formulation.

If desired, tablet unit dosage forms of the disclosed compositions may be coated by standard aqueous or nonaqueous techniques. In one embodiment, coating with hydroxypropylmethylcellulose (HPMC) is employed.

Specific pharmaceutically acceptable carriers are described in U.S. Pat. No. 4,401,663; European Patent Application No. 089710; and European Patent Application No. 0068592, which are incorporated herein by reference. The amount of a carrier employed in conjunction with the eszopiclone is sufficient to provide practical quantity of material per unit dose of eszopiclone.

Exemplary pharmaceutically acceptable carriers for systemic administration, which may be incorporated in the composition of the present disclosure, include sugar, starches, cellulose, vegetable oils, mineral oils, buffers, polyols, alginic acid and the like. Exemplary carriers for parenteral administration include propylene glycol, pyrrolidone, ethyl oleate, aqueous ethanol and combinations thereof. Still other representative carriers include acacia, agar, alginates, hydroxyalkylcellulose, hydroxypropyl methylcellulose, carboxymethylcellulose, carboxymethylcellulose sodium, carrageenan, powdered cellulose, guar gum, cholesterol, gelatin, gum agar, gum arabic, gum karaya, gum ghatti, locust bean gum, octoxynol-9, oleyl alcohol, pectin, poly(acrylic acid) and its homologs, polyethylene glycol, polyvinyl alcohol, polyacrylamide, sodium lauryl sulfate, poly(ethylene oxide), polyvinylpyrrolidone, glycol monostearate, propylene glycol monostearate, xanthan gum, tragacanth, sorbitan esters, stearyl alcohol, starch and its modifications. Suitable ranges vary from about 1% to about 99.5% by weight, such as from about 20% to 80% by weight of the total composition.

In certain embodiments, a unit dosage form can include 1% or more by weight of eszopiclone, such as 2%, 3% or more by weight of eszopiclone. In certain aspects, a unit dosage form may contain less than 1% by weight of eszopiclone, e.g. 0.9% by weight, 0.8% by weight, 0.7% by weight, 0.6% by weight, 0.5% by weight, 0.4% by weight, 0.3% by weight, 0.2% by weight or 0.1% by weight of eszopiclone.

In various alternative embodiments, the unit dosage form can include eszopiclone that is administered by sustained release means or by a sustained release component. It is noted that sustained release embodiments can contain an amount of eszopiclone that would be considered a sedative dose if all of the compound were formulated as an instant release component. Exemplary sustained release formulations and components include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide sustained release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable sustained release formulations known to those of ordinary skill in the art, including those described herein, can be readily selected for use with eszopiclone and other active ingredients disclosed herein.

The unit dosage form of the present disclosure can also include a sustained release dosage form eszopiclone and an instantaneous release dosage form of this compound. The sustained release and instantaneous release components of the dosage form can be combined in a single compartment or they can be each in a separate compartment of the unit dosage form. When the sustained and instantaneous dosage components are in separate components, the compartments can be separated by substantially any means, e.g., isolated compartments separated by a physical barrier, particles of differing size, etc.

For example, in one embodiment, the separate compartments are particles of eszopiclone of a first size and a second size, respectively. The particles of the first size and the particles of the second size release said compound into plasma at a first rate and a second rate. The first rate and the second rate can be different rates. Such drug particles suitably may include a variety of excipients (e.g. to form granules or microspheres) in addition to drug agent.

In various alternative embodiments, the sustained release dosage component includes at least a portion of the active ingredient entrapped within a matrix, e.g., a polymeric matrix. The polymer can be natural or synthetic. In some embodiments, at least 50%, such as at least 75% or at least 90% of the active compound in the sustained release component is entrapped within a polymeric matrix.

The sustained release component of the unit dosage form of the present disclosure can release the active component over a broad range of [eszopiclone]/time ratios. In general, compositions of the present disclosure provide a plasma concentration of eszopiclone correlating to an antianxiety dose for at least about 6 hours, such as at least about 8 hours, at least about 10 hours, at least about 12 hours or at least about 14 hours. In various embodiments, the dosage can provide a plasma concentration of eszopiclone that is anxiolytic without being moderately sedative during the period of anxiolysis induced by the eszopiclone.

For clarity of illustration the sustained release unit dosage forms of the present disclosure are described in the context of orally administered dosage forms. The present disclosure thus encompasses single unit dosage forms suitable for oral administration such as, but not limited to, tablets, capsules, gelcaps, and caplets that are adapted for controlled-release. Other unit dosage forms are within the scope of the present invention.

Sustained release pharmaceutical products have a common goal of improving drug therapy over that achieved by their non-sustained release counterparts. Ideally, the use of an optimally designed sustained release preparation in medical treatment is characterized by a minimum of drug substance being employed to cure or control the condition in a minimum amount of time. Advantages of sustained release formulations include extended activity of the drug, reduced dosage frequency, and increased patient compliance. In addition, sustained release formulations can be used to affect the time of onset of action or other characteristics, such as blood levels of the drug, and can thus affect the occurrence of side (e.g., adverse) effects.

Most sustained release formulations are designed to initially release an amount of drug (active ingredient) that promptly produces the desired therapeutic effect, and gradually and continually release of further amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Sustained release of an active ingredient can be stimulated by various conditions including, but not limited to, pH, temperature, enzymes, water, or other physiological conditions or compounds.

In an exemplary embodiment, the present dosage form can include a mechanically disruptible release coat providing a sustained release mechanism. An exemplary formulation includes a core containing a drug and a swelling agent, coated with a water-insoluble but permeable polymer, see, Ueda et al. in Journal of Drug Targeting (1994) 2: 35. After the device is orally administered, water permeates into the core, which hydrates and swells. The stress caused by the swelling disrupts the coating to enable drug release. Different fillers can be used, including an effervescent agent, which can be filled into capsules and coated with water-insoluble polymers.

In another embodiment, the sustained release formulation includes an osmotic release component. Santus et al., Journal of Controlled Release (1995) 35: 1 reviewed the osmotic drug release literature. An exemplary osmotic formulation is one in which osmotic pressure exerts a stress on a membrane, resulting in a gradual release of the drug.

Another type of delivery system relies on hydration or erosion to provide sustained release of eszopiclone. Thus, in an exemplary embodiment the unit dosage form consists of a water insoluble capsule filled with a drug plugged with a hydrogel and covered with a water-soluble cap. After the capsule is orally administered the cap dissolves and the hydrogel plug becomes fully hydrated after a certain time and is expelled, thereby permitting a rapid and complete release of the drug. Such a device referred to as the Pulsincap™ device was disclosed by Scherer, see Pharma. J. (1991) 247: 138. An alternative pulsatile drug release system is described by Krogel et al. in Pharmaceutical Research (1998) 15: 474, using an erodible plug formed by compression or from a melt as a closure to an impermeable capsule body.

Yet another sustained release delivery system based on hydration and erosion is that described by Pozzi et al. in Journal of Controlled Release (1994) 31: 99. The device is a solid core coated with a hydrophobic-surfactant layer, applied as an aqueous dispersion, to which a hydrosoluble polymer can be added to improve adhesion to the core. The coating rehydrates and redisperses in an aqueous environment in a time proportional to the thickness of the film. Thus the coat has been designed to be completely removed after a pre-determined lag time depending on the coat thickness. The different physiological and chemical environments within the gastrointestinal tract are not expected to alter significantly the release time. In another exemplary embodiment, the eszopiclone is incorporated in this type of sustained release formulation.

A further sustained release system comprises a solid core of drug and an organic acid such as succinic acid and coated with a thick coat of Eudragit R S (Narisawa et al, Pharm. Res. (1994) 11: 111 and Narisawa et al. International Journal of Pharmaceutics (1997) 148: 85). Eudragit R S is a copolymer synthesized from acrylic and methacrylic acid esters, which forms a film that is water insoluble with low permeability. On full hydration, water gradually penetrates the membrane into the core and dissolves the organic acid. The resulting polymer/acid interaction induces a structural change in the coating film, increasing permeability, which enhances the drug release. The present disclosure also provides a sustained release component according to this design, or using a polymer with a behavior analogous to that of Eudragit R S.

In another embodiment, the unit dosage formulation includes a sustained release component such as that disclosed by Ishibashi et al., International Journal of Pharmaceutics (1998) 168: 31. In this embodiment, a blend of eszopiclone and organic acid forms solid cores loaded into gelatin capsules. The capsule can be coated with three different polymeric layers; an inner layer consisting of cationic polymer dissolving in acidic fluid, a water-soluble intermediate layer, and an outer layer consisting of enteric materials dissolving at pH above 5. The intermediate layer serves to prevent direct contact between the inner and outer layers. This formulation essentially prevents drug release in the stomach by the outer polymeric layer, after gastric emptying the outer and intermediate layers quickly dissolve but the inner polymeric layer remains to prevent drug release in the intestine, and then when the pH inside the capsule gradually decreases with dissolution of the organic acid and the inner polymeric layer is dissolved by the acidic fluid, the drug content is quickly released.

In yet another embodiment, the enteric behavior of some polymers, whereby the delay is dependent upon gastric residence time, is utilized to obtain sustained release of eszopiclone. Devices of this kind, which may comprise tablets capsules, spheroids and beads, can be coated with polymers that dissolve only in a medium of pH 5 or higher. The coated core survives the low pH in the stomach and releases its contents rapidly in the alkaline environment of the upper part of the intestine.

In still another embodiment, the particles of eszopiclone include a population of particles that are within a controlled range of sizes. For example, in a selected unit dosage form at least about 80% or at least about 90% of the eszopiclone is present in particles of a size less than or equal to about 50 μm.

In another exemplary embodiment, no more than about 20% or no more than about 10% of the eszopiclone is present in particles of a size greater than about 50 μm. Methods of making small particles, and particles within a defined size range are well known in the art. See, for example, Lieberman et al., “Pharmaceutical Dosage Forms: Tablets,” Vol. 2, Marcel Dekker, p. 114 (1989); Lachman et al., “Theory and Practice of Industrial Pharmacy”, Lea & Febiger, p. 37 (1985); and McCabe et al, “Size reduction, in Unit Operations of Chemical Engineering”, McGraw-Hill, p. 809 (1967). Milling units that are suitable for eszopiclone milling include hammer mills, cutting mills, roller mills, and jet mills.

In a further embodiment, at least a portion of the eszopiclone is present in a crystalline state.

In a further aspect, the unit dosage form can include one or more therapeutic agent in addition to eszopiclone. Exemplary agents include benzodiazepines; 5-HT_{1A} ligands; 5-HT_{1B} ligands; 5-HT_{1D} ligands; mGluR2A agonists; mGluR5 antagonists; antipsychotics; NKI receptor antagonists; antidepressants; serotonin reuptake inhibitors; GABA_{B} ligands; mood stabilizers; antiepileptic agents; sodium channel blockers; N-type channel ligands; agents to treat sleep disorders including sleep apnea and restless leg syndrome; agents to treat mood disorders such as depression, bipolar, dysthymia, premenstrual syndrome and perimenopausal symptoms; agents for treatment of pain; anesthetics; agents to treat extrapyramidal symptoms such as antiparkinsons, tardive diskinesias, dystonia, Huntington's, myoclonus (restless leg), tics (tourettes) and the like; agents to treat neurodegenerative and neuromuscular disorders such as MS, ALS, and Creutzfeldt Jacob; agents to relax muscle tension, and antispastic drugs including treatment of stuttering, premature ejaculation, nausea and vomiting.

Exemplary benzodiazepines may include but are not limited to adinazolam, alprazolam, bromazepam, clonazepam, chlorazepate, chlordiazepoxide, diazepam, estazolam, flurazepam, balezepam, lorazepam, midazolam, nitrazepam, oxazepam, quazepam, temazepam, triazolam and equivalents thereof.

Exemplary 5-HT_{1A} and/or 5HT_{1B} ligands may include but are not limited to buspirone, alnespirone, elzasonan, ipsapirone, gepirone and equivalents thereof.

Exemplary mGluR 2 agonists include, but are not limited to, (1S,3R)-1-aminocyclopentane-1,3-dicarboxylic acid, (2S,3S,4S)alpha-(carboxycyclopropyl)glycine, and 3,5-dihydroxyphenylglycine.

Exemplary antidepressants include, but are not limited to, maprotiline, amitriptyline, clomipramine, desipramine, doxepin, imipramine, nortryptyline, protriptyline, trimipramine, SSRIs and SNRIs such as fluoxetine, paroxetine, citalopram, escitalopram, sertraline, venlafaxine, fluoxamine, and reboxetine.

Exemplary antipsychotics include, but are not limited to, clozapine, risperidone, quetiapine, olanzapine, amisulpride, sulpiride, zotepine, chlorpromazine, haloperidol, ziprasidone, and sertindole.

Exemplary mood stabilizers include, but are not limited to, valproic acid (valproate) and its derivative (e.g. divalproex), lamotrigine, lithium, verapamil, carbamazepine and gabapentin.

With regard to the agents other than eszopiclone, the appropriate dose regimen, the amount of each dose of an active agent administered, and the specific intervals between doses of each active agent will depend upon the subject being treated, the specific active agent being administered and the nature and severity of the specific disorder or condition being treated.

### Methods

The present invention also provides methods for the treatment and prophylaxis of anxiety. The method uses low dosages of eszopiclone within a defined range to provide anxiolysis. In an alternative embodiment, the dosage is sufficiently low that the eszopiclone remains at a subsedative plasma concentration during the entire transit of the eszopiclone through the subject's body. In a further embodiment, the dosage is sufficiently low that the eszopiclone remains at a subsedative plasma concentration during a substantial portion of the transit of the eszopiclone through the subject's body.

Eszopiclone, within the controlled dosage range disclosed herein exerts an unexpected and clinically determinable anxiolytic effect on mammalian subjects. The range of anxiolytic dosages and concentrations of the present disclosure are not disclosed in the art. Moreover, though the art recognizes zopiclone and eszopiclone as agents useful for treating various disorders, the art discloses methods that rely on use of the active agent within broad dosage ranges-including dosages at least an order of magnitude higher than those disclosed herein. The prior art ranges, furthermore, encompass known sedative and hypnotic dosages.

Accordingly, the present disclosure provides a method of treating anxiety in a subject, such as a human subject. The method includes administering to a subject in need of treatment for or prophylaxis of anxiety, a unit dosage form comprising an amount of eszopiclone sufficient to induce anxiolysis. The dosage used is generally preferred to be insufficient to induce moderate sedation in the subject.

Exemplary types of anxiety treatable according to the instant method include panic attack, agoraphobia, acute stress disorder, specific phobia, panic disorder, psychoactive substance anxiety disorder, organic anxiety disorder, obsessive-compulsive anxiety disorder, posttraumatic stress disorder and generalized anxiety disorder.

The methods herein include administering to a subject identified as in need of anxiolytic treatment an effective amount of eszopiclone. Identifying a subject in need of such treatment can be in the judgment of a subject or a health care professional and can be subjective (e.g. opinion) or objective (e.g. measurable by a test or diagnostic method).

Methods of the invention may suitably further include confirming the efficacy of treatment. Such confirmation by be performed e.g. by a health care professional).

As discussed above, it has also been discovered that administration of low dosages of eszopiclone to a subject at times other than at the subject's bedtime, exert an unexpected anxiolytic activity. Eszopiclone is used principally as a sleep aid in dosages that provide at least moderate sedation. Moreover, when eszopiclone is used to treat or ameliorate a disease or condition other than insomnia (e.g., schizophrenia, convulsions, etc.), the dosages administered are within a range sufficient to induce at least moderate sedation in the subject to whom eszopiclone is administered, and these dosages are generally administered at the subjects bedtime. The use of dosages of eszopiclone set forth herein, administered to the subject at a time other than the subject's bedtime, e.g., upon arising, prior to or following breakfast or at scheduled intervals during the subject's waking hours including prior to 5:00 pm, 6:00 pm, 7:00 pm or 8:00 pm, is not suggested in the art In an exemplary embodiment, in which an otherwise healthy adult human is treated for anxiety, the method includes administering to the subject an antianxiety dose of eszopiclone reaching a maximum plasma concentration (Cₘₐₓ) in the subject of from about 0.1 ng/mL to about 25 ng/mL. The Cₘₐₓ can be from about 0.5 ng/mL to about 20 ng/mL, such as from about 1 ng/mL to about 10 ng/mL of eszopiclone.

An exemplary method includes administering a dose of eszopiclone in an amount sufficient to achieve a maximum plasma concentration (Cₘₐₓ) of not more than about 20 ng/mL of said compound, such as not more than about 8 ng/mL of said compound.

In still a further embodiment, a method includes administering to the subject a unit dose of eszopiclone in an amount sufficient to provide a dose of less than about 4 mg/70 kg drug/patient weight, such as from about 0.25 mg/70 kg to about 0.9 mg/70 kg drug/patient weight, or from about 0.5 mg/70 kg to about 0.9 mg/70 kg drug/patient weight. In another exemplary embodiment, the dose can be less than about 1 mg/70 kg drug/patient weight.

In one aspect, the dose administered to a healthy 70 kg adult subject preferably can be from about 0.4 mg to about 0.9 mg of eszopiclone. Preferably, the healthy subject does not require treatment for any one or more of the following conditions, insomnia, epilepsy, muscle spasms, stuttering, schizophrenia, or chronic pain. More preferably, the subject does not require treatment for any condition other than anxiety, which could be treated by the dosages of eszopiclone set forth herein. Equivalent, proportional dosages (less or more eszopiclone, respectively) for lighter and heavier subjects as well as subjects suffering from conditions in which a lower or higher dosage is implicated are included within this invention.

An exemplary method uses a dose of eszopiclone that correlates in a human subject to an anxiolytic dosage determined by the rhesus monkey conflict procedure reported by Rowlett et al. (Psychopharmacology (2006) 184:201-211).

In yet another alternative embodiment, the dose of eszopiclone is administered to the subject using a unit dosage form, such as those illustrated in the preceding sections. However, it is understood that the amount of the eszopiclone administered to a subject will be determined by a physician, in the light of the relevant circumstances including the frequency of the condition to be treated, the age, weight, and response of the individual subject, the severity of the patient's symptoms, and the chosen route of administration, and therefore the above dosage ranges are not intended to limit the scope of the invention in any way. In most situations, however, the dosage administered is preferably a subsedative dosage.

While the present compounds can be administered orally to humans susceptible to or suffering from anxiety, the compounds may also be administered by a variety of other routes such as the transdermal, parenterally, subcutaneous, intranasal, intramuscular and intravenous routes. Such formulations may be designed to provide delayed or controlled release using formulation techniques which are known in the art.

In general, the doses of eszopiclone of use in the presently disclosed methods, when used as either a single active agent or when used in combination with another active agent, will be administered to a subject in single or divided doses. The eszopiclone may be administered on a regimen of up to about 6 times per day, such as 1, 2 3 or 4 times per day. Variations may nevertheless occur depending upon the subject being treated and the individual response to the treatment, as well as on the type of pharmaceutical formulation chosen and the time period and interval at which such administration is carried out. In some instances, dosage levels below the lower limit of the aforesaid ranges may be more than adequate, while in other cases larger doses may be employed to achieve the desired effect. When either smaller or larger doses are utilized, in most cases each of these dosages is preferably subsedative.

In other aspects, the racemate zopiclone can be utilized herein in the same manner as described for the S-isomer eszopiclone. However, it is recognized that use of eszopiclone can provide advantages over use of the racemate zopiclone and thereafter use of eszopiclone will be preferred for many applications.

All documents mentioned herein are specifically incorporated by reference herein in their entirety.

The materials, methods and devices of the present disclosure are further illustrated by the examples that follow. These examples are offered to illustrate, but not to limit the claimed invention.

### EXAMPLE 1

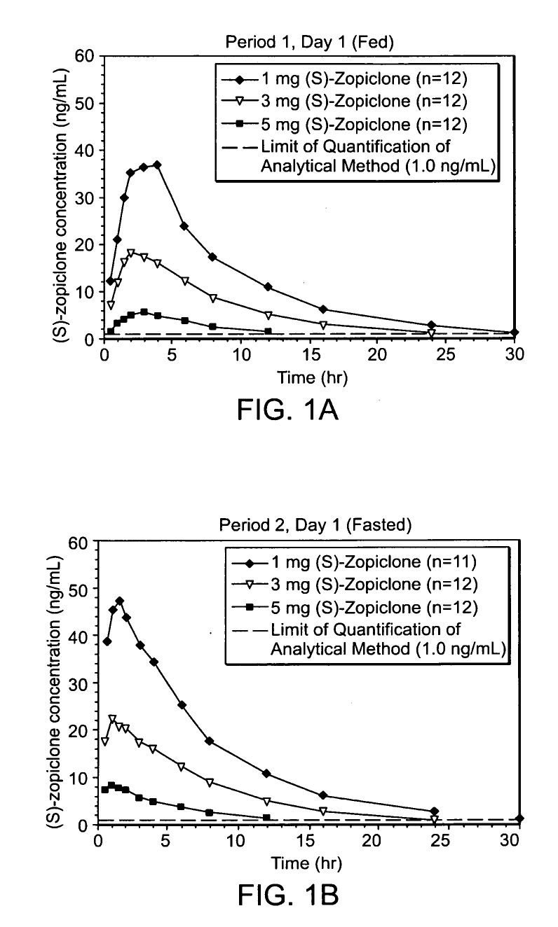

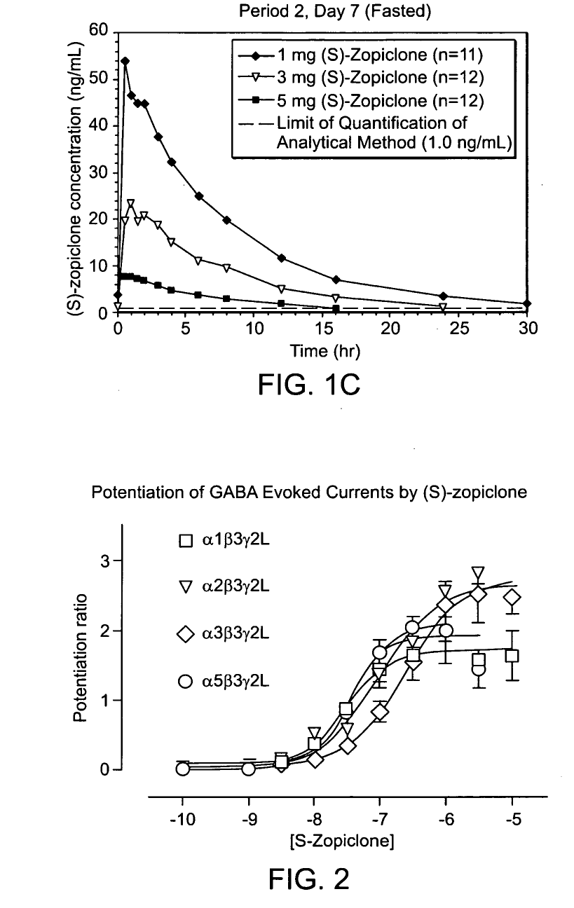

A clinical study of eszopiclone in healthy volunteers was performed using art-recognized methodology. The plasma concentration curves for various doses of eszopiclone are shown in FIG. 1.

### EXAMPLE 2

### Concentration-Dependent Potentiation of GABA Evoked Currents By (S)-Zopiclone.

The GABA-A receptor is gated by GABA, and ligands at the benzodiazepine site potentiate GABA-evoked currents. It is well-established in the scientific literature that classical benzodiazepines potentiate GABA evoked chloride currents through GABA-A receptors. The efficacies of classical benzodiazepines can be described as “full modulators” referring to their ability to increase the chloride currents induced by sub-maximal GABA concentrations. The action of (S)-zopiclone on GABA-A receptors was evaluated by examining their effects on electrophysiological recordings in a recombinant expression system.

Recombinant GABA-A receptors were expressed in Xenopus oocytes by injecting cDNA encoding the human β3, γ2L, and each of α1, α2, α3, or α5 subunits in turn to assemble functional channels. Channel activity was measured using two-electrode voltage clamp in whole cell configuration on a robotic workstation. Oocytes were held at resting membrane potential of −80 mV and solutions were exchanged by perfusion using an automated ion channel screening system (Roboocyte, Multi channel Systems, Germany). A full concentration response curve for each subtype was generated to determine the EC₁₀ of GABA. The potentiation ratio was then measured as the fold-increase in current generated by a (S)-zopiclone plus an EC₁₀ of GABA, compared to the current generated by just the EC₁₀ of GABA alone. These potentiation ratios were measured at a range of (S)-zopiclone concentrations to generate a subsequent concentration response curve at each of the subtypes using between 2 and 7 oocytes. Parameters of a sigmoidal dose-response equation were adjusted by non-linear fitting of log EC₅₀ using a sigmoidal dose response relationship (GraphPad Prism 4). Further experimental details are reported in the description of FIG. 2.

In experiments investigating the effects of (S)-zopiclone on GABA evoked currents in human recombinant GABA-A receptors expressed in Xenopus oocytes, (S)-zopiclone potentiated the effects of GABA at all GABA-A receptor subtypes in a concentration-dependent manner, with maximal effects seen at a concentration of 1 μM as shown in FIG. 2. The EC₅₀ values for α1-, α2-, α3-, and α5-containing GABAA receptors were 30, 100, 250, and 40 nM, respectively, and the maximal fold-increases (potentiation) induced by (S)-zopiclone were 1.7, 2.7, 2.7, and 2.1, respectively.

For all experiments, human cDNA encoding each subunit was injected in oocytes in a stoichiometry of 1:1:3 (alpha, beta, gamma subunits). Approximately 24 hours later, GABA evoked currents (using a concentration of GABA equal to the EC₁₀) were recorded from oocytes using a two-electrode voltage clamp on a robotic oocyte platform and a constant voltage of −80 mV. GABA was applied to the oocytes via a perfusion system for 20 seconds and then removed. An inward deflection of the current trace was recorded and subsequently compared to the responses resulting from applying the same concentration of GABA plus varying concentrations of the test compound [(S)-zopiclone]. A doubling of the GABA evoked current is represented as a potentiation ratio of 1.0.

### EXAMPLE 3

In vivo efficacy of (S)-zopiclone in a rhesus conflict model of anxiety matches the efficacy of classical benzodiazepines used clinically for the treatment of anxiety.

Conflict procedures in animals are used to study mechanisms underlying the anxiolytic effects of benzodiazepines (Rowlett J K et al., Psychopharmacology (2006) 184:201-211). The potencies of 10 classical benzodiazepines (alprazolam, flunitrazepam, clonazepam, nitrazepam, lorazepam, bromazepam, diazepam, flurazepam, clorazepate, chlordiazepoxide) in a rhesus conflict procedure have been shown to correlate with potencies for therapeutic effects in humans (Rowlett J K et al., 2006). The effects of each benzodiazepine in rhesus conflict procedure is to increase suppressed responding at doses lower than the doses that ultimately decrease rates of non-suppressed responding. This characteristic profile in the rhesus conflict procedure contrasts dramatically with the effects of non-benzodiazepine hypnotics zolpidem and zaleplon, and also contrasts to the effects of GABA-A ligands with subtype efficacies different from classical benzodiazepines, such as flumazenil and CL218,872 (Rowlett J K et al., 2006).

However unexpectedly based on its non-benzodiazepine chemical class, (S)-zopiclone produces anti-conflict effects in the rhesus model indistinguishable from the classical benzodiazepines' effects. (S)-zopiclone was shown to produce its anxiolytic effects at lower doses than those used clinically for hypnotic therapy (see below).

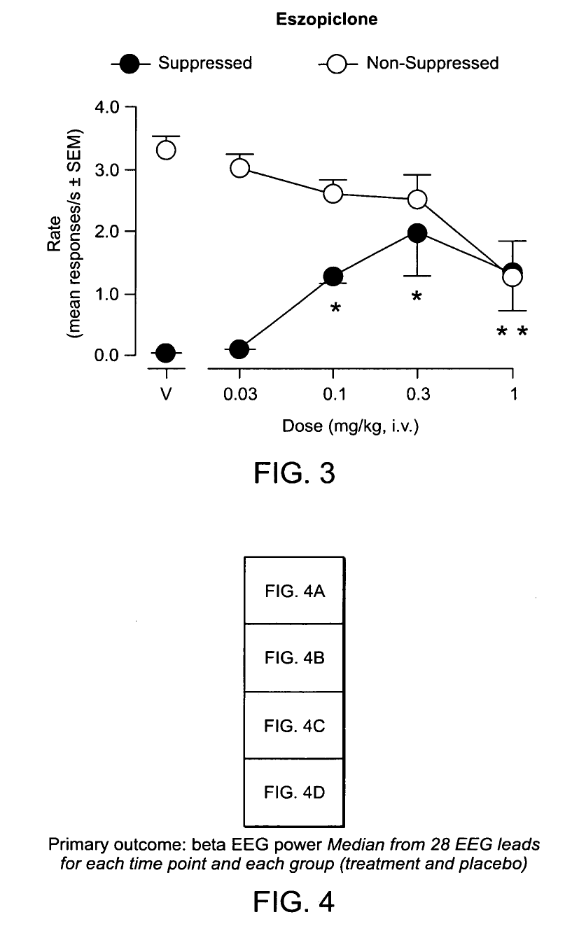

The 10 classical benzodiazepines studied in Rowlett J K et al. (2006) demonstrate a correlation between therapeutic (oral once per day) doses in humans and the ED₅₀ values for the suppressed responding after i.v. administration to rhesus monkeys. This correlation was used to estimate the therapeutic dose in humans for anxiolytic effects. The ED₅₀ of the suppressed responding in FIG. 3 is 0.07 mg/kg i.v., indicating a 50% anxiolytic effect at that dose i.v. in the rhesus procedure. Using this ED₅₀ in the regression analysis of Rowlett J K et al. (2006), an expected human oral dose of 0.9 mg is obtained. This dose is below the hypnotic doses (i.e., it is subsedative) used clinically in humans (1, 2, and 3 mg), further supporting the validity of the correspondence between the efficacy profiles of classical benzodiazepines and (S)-zopiclone.

The potencies of eszopiclone to alter suppressed and non-suppressed responding were estimated by calculating the dose that engendered 5% to 95% of the maximum effect (EDₓ, where x=5-95%). The EDₓ for suppressed responding was obtained by dividing rates of suppressed responding for individual monkeys by the average maximum increase in response rate (i.e., the peak effect) and multiplying the value by 100. Doses on the ascending limb of the dose-response function were used to calculate the EDₓ by log-linear regression analysis. The EDₓ for non-suppressed responding was obtained by dividing rates of non-suppressed responding for individual monkeys by the average maximum decrease in response rate (this corresponded to the highest dose tested) and multiplying by 100. The EDₓ values for non-suppressed responding were calculated by log-linear regression analysis. All potencies were expressed as the mean±SEM for n=4 monkeys.

A linear relationship between relative potency to induce clinical effects in humans and relative potency to increase suppressed responding or decrease non-suppressed responding in monkeys has been determined previously for 10 standard benzodiazepines (Rowlett et al., 2006). The potency relationships were described by the equations y=0.672x−0.174 (suppressed responding) and y=0.583x−0.175 (non-suppressed responding), in which y=log₁₀ value of the potency of test compound relative to diazepam in humans, and x=log₁₀ value of the corresponding potency in monkeys (Rowlett et al., 2006). Using this equation, the potency of eszopiclone to induce effects in humans relative to diazepam was calculated. The first step included calculating relative potencies in the monkey conflict procedure by dividing the ED₅₀ values for eszopiclone by the ED₅₀ values for diazepam (previously published in Rowlett et al., 2006). Next, these relative potencies were converted to log₁₀ values and were entered into the linear equations in order to obtain a predicted relative potency for clinical doses. To calculate estimated clinical doses in humans, the lowest (2 mg) and highest (10 mg) recommended clinical doses of diazepam for adults were used. Results are tabulated below.

TABLE 1
Eszopiclone potencies (EDx)* to increase rates of suppressed responding
(N = 4

| x (% maximum increase) | ED (mean mg/kg, i.v.) | SEM |
|---|---|---|
| 5 | 0.029 | 0.001 |
| 10 | 0.032 | 0.001 |
| 15 | 0.036 | 0.001 |
| 20 | 0.040 | 0.001 |
| 25 | 0.044 | 0.001 |
| 30 | 0.048 | 0.001 |
| 35 | 0.054 | 0.002 |
| 40 | 0.059 | 0.002 |
| 45 | 0.066 | 0.003 |
| 50 | 0.073 | 0.004 |
| 55 | 0.081 | 0.005 |
| 60 | 0.090 | 0.006 |
| 65 | 0.100 | 0.008 |
| 70 | 0.110 | 0.009 |
| 75 | 0.123 | 0.011 |
| 80 | 0.136 | 0.014 |
| 85 | 0.151 | 0.017 |
| 90 | 0.168 | 0.020 |
| 95 | 0.186 | 0.024 |

*EDx is the dose resulting in x % of the maximum effect, computed by linear regression analysis for individual monkeys, then averaged with SEM computed.

TABLE 2
Calculation of apparent anxiolytic and sedative doses for eszopiclone

| | | | Y = 0.672x − 0.174Y |
|---|---|---|---|
| Suppressed responding | | | (eszopiclone |
| Eszopiclone ED50 | Ratio | Log10(Ratio) | relative potency) |
| 0.07 mg/kg | 0.64 | −0.19 | −0.34 |
| Diazepam ED50 | | | 10\hat{ }Y= |
| 0.11 mg/kg | | | 0.46 |

Minimum recommended dose of diazepam = 2 mg
Estimated anxiolytic dose for eszopiclone = 0.9 mg

| Non-suppressed | | | Y = 0.583x − 0.175Y |
|---|---|---|---|
| responding | | | (eszopiclone |
| Eszopiclone ED50 | Ratio | Log10(Ratio) | relative potency) |
| 0.74 mg/kg | 0.31 | −0.51 | −0.47 |
| Diazepam ED50 | | | 10\hat{ }Y= |
| 1.65 mg/kg | | | 0.34 |

Maximum recommended dose of diazepam = 10 mg
Estimated sedative dose for eszopiclone = 3.4 mg

### EXAMPLE 4

### EEG Study

A study was conducted to evaluate the pharmacodynamic (quantitative wake electroencephalogram (qEEG) and auditory P300 (evoked potentials (EPs)) of four doses of eszopiclone, relative to placebo, in healthy adult male human subjects. The study was single-blind, placebo-controlled, ascending, single dose. Subjects received single doses of immediate release formulations that contained 0.3 mg of eszopiclone, 0.6 mg of eszopiclone, 0.9 mg of eszopiclone or 2.0 mg of eszopiclone.

### 1. Primary Outcome: Beta EEG Power

### Interkinetic Analysis (Change From Placebo)

### Beta EEG Activity

Changes in beta EEG activity are interpreted as indicating an anxiolytic effect (Ansseau et al, Neuropsychobiology 1984, 12(4):255-9; Buchsbaum et al, Biol. Psychiatry, 1985, 20(8):832-42; Macher et al, Human psychopharmacology, 1990 (eds I Hindmarch and P D Stonier), Vol 3, 10-2; Mandema and Danhof, Clin Pharmacokinet, 1992, 23(3):191-215.

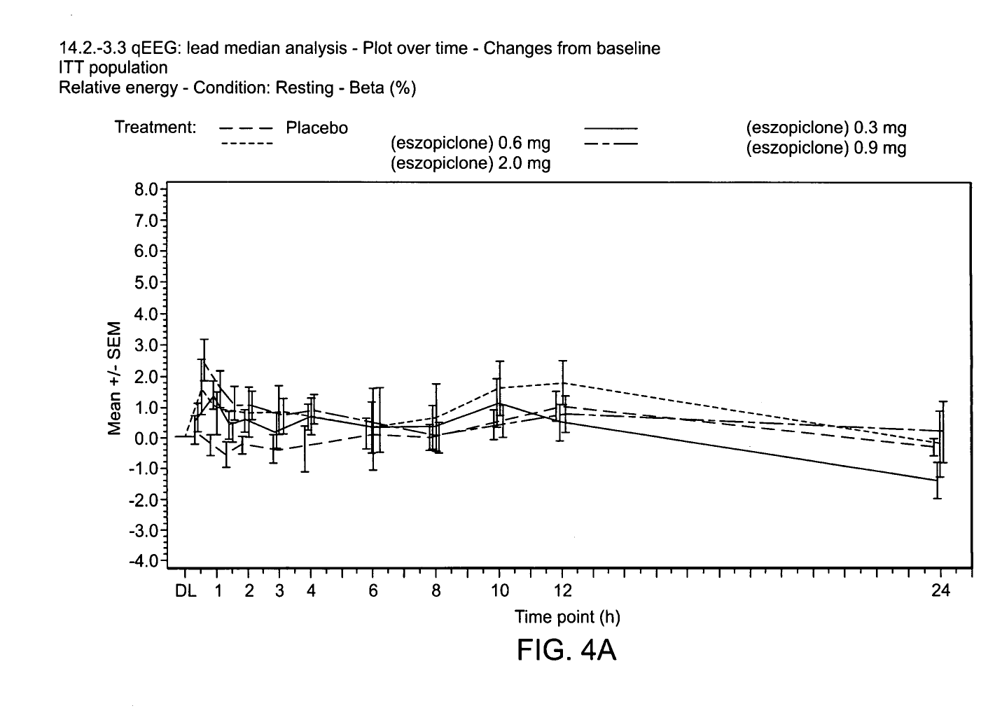

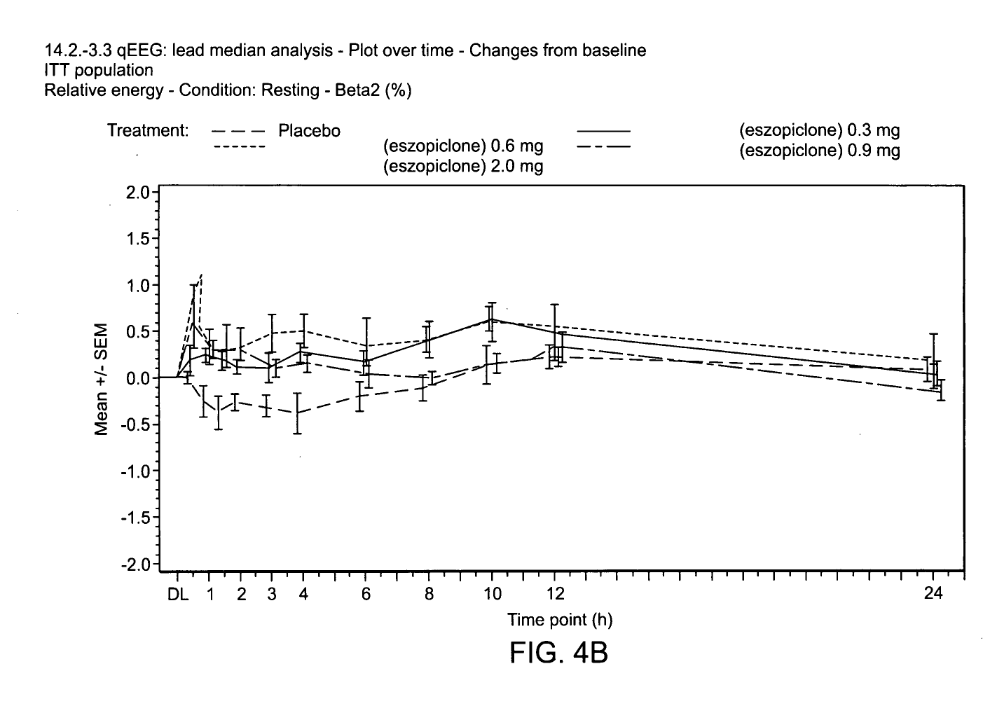

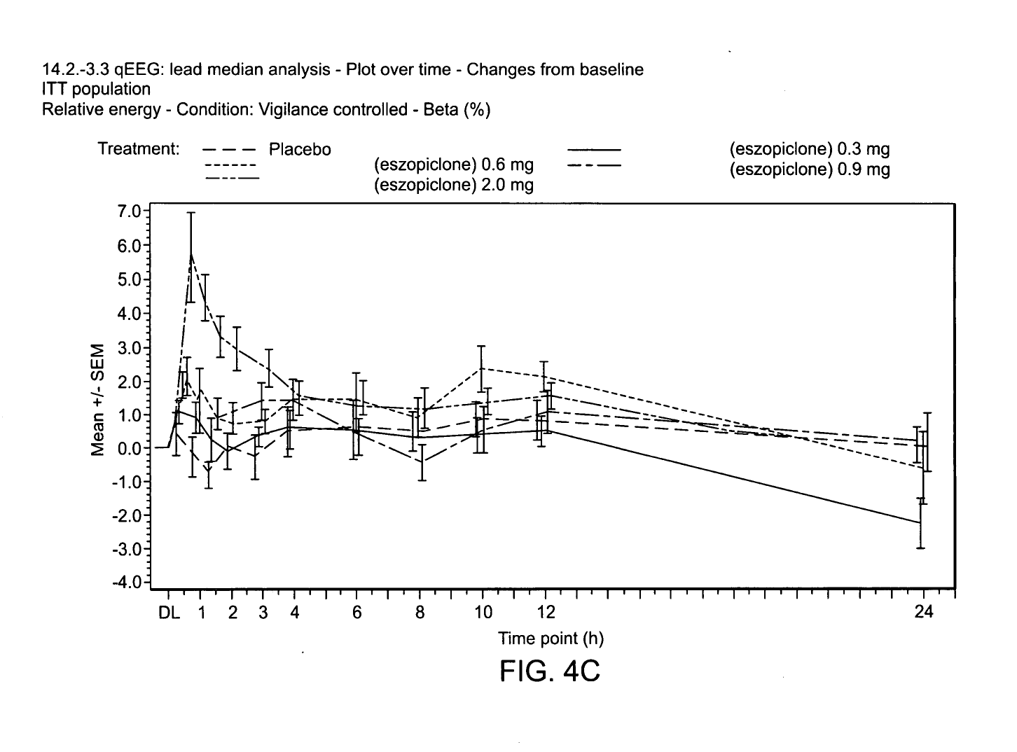

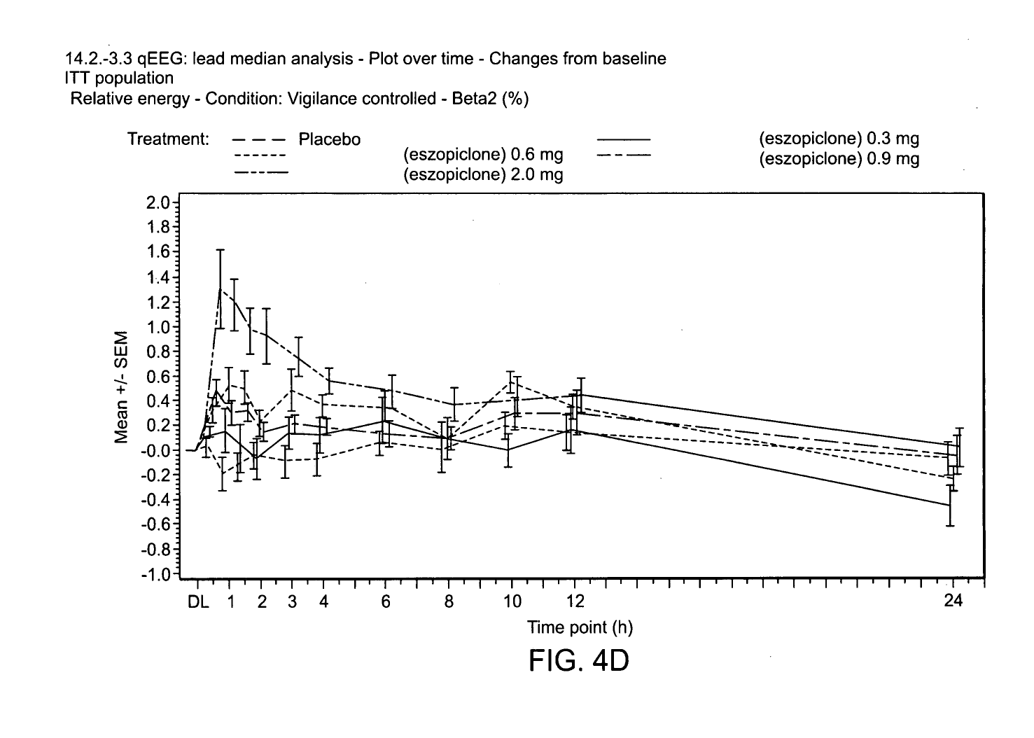

In this study, statistical EEG maps were obtained comparing EEG power values for each dose of eszopiclone versus placebo. Results indicate that eszopiclone induces a significant increase in beta band and/or one or several beta sub bands and that these changes are dose-dependent (strongest for the highest dose and more attenuated when the dose decreases). The dose effect is further illustrated in the graphically depicted results of FIG. 4 which follows.

### 2. Secondary Outcomes: Other EEG Parameters (Delta, Alpha and Theta Bands)

### Interkinetic Analysis (Change From Placebo)

### Delta EEG Activity

Classically, delta activity in pharmaco-EEG indicates a sedative effect of the drug under study. Delta EEG activity is clearly modified when the sedative drug effect has reached a high threshold. The results from this study indicate that only the highest dose of eszopiclone induces a significant sedative effect whereas the other doses do not have this side effect.

Conclusions regarding a possible sedative effect or a trend to impair the subject's arousal should also include analyses of a drug's effects on alpha EEG activity, which reflect decreases in subject's arousal (pro sedative effect).

### Alpha EEG Activity

Similar to changes observed in delta EEG activity, alpha EEG activity was mainly influenced (decreased) by the highest dose of eszopiclone (2.0 mg). These results indicate that the highest dose of eszopiclone (2.0 mg) induces a significant impairment in the subject's arousal and possibly a sedative effect. Doses below 2.0 mg did not exhibit major effects on arousal.

### Theta EEG Activity

The theta rhythm is regarded as a basal rhythm associated with cognitive function and corticohippocampal interactions, which functionally mediate processes related to emotional activation. In this study, all doses of eszopiclone induced a significant decrease in theta power (absolute as well as relative) with a dose dependence, as displayed in the graph below. In previous studies, it has been shown that theta EEG activity was increased in panic disorder patients (Knott et al, J. Anxiety Disorder, 1997, 11(4):365-76) and in healthy subjects experimenting an anxiety induced by emotiogenic films (Aftanas et al, 2006, Neurosci Behav Physiol., 2006, 36(2): 119-30). Thus, consistent with changes observed in beta EEG activity, observed decreases in theta EEG activity observed after administration of eszopiclone support the anxiolytic effect of the compound.

### Conclusion

Consistent with classical interpretation of beta EEG activity increases related to a drug effect (Ansseau et al, 1984; Buchsbaum et al, 1985; Macher et al, 1990; Mandema and Danhof, 1992), it can be concluded that all doses of eszopiclone administered in this study demonstrated antidepressant effects in humans. In addition, observed decreases in theta EEG activity are indicative of anxiolytic efficacy based on reports of increased theta EEG activity in both patients with anxiety disorder (Knott et al, 1997) as well as induced anxiety models in healthy normals (Aftanas et al, 2006).

With regard to the potential for sedation, increase in delta and decrease in alpha EEG activities induced by the highest dose obviously suggest that this dose has a pro-sedative effect or at least, significantly impairs the subject's arousal level. However, doses below 2.0 mg did not exhibit significant effects on arousal or sedation. Taken together, results from qEEG analyses (beta, alpha delta and theta bands) show that the administered exposures of eszopiclone below 2.0 mg are particularly appropriate for development as an anxiolytic in the absence of sedative effects.

The above-disclosed subject matter is to be considered illustrative, and not restrictive, and the appended claims are intended to cover all such modifications, enhancements, and other embodiments that fall within the true scope of the present invention. For example, it is noted that certain components in the exemplary embodiments described herein as having a particular function or as being present in a particular form are illustrative and it is noted that such components can perform additional functions or be present in different forms. Thus, to the maximum extent allowed by law, the scope of the present invention is to be determined by the broadest permissible interpretation of the following claims and their equivalents, and shall not be restricted or limited by the foregoing detailed description.

1. A unit dosage form comprising up to 0.9 mg of a compound having the structure: 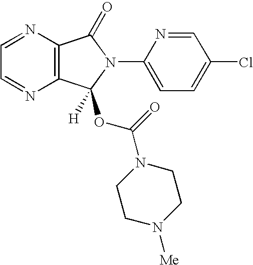
or a pharmaceutically acceptable salt, solvate, hydrate, polymorph, or prodrug thereof.

2. A modified unit dosage form comprising a compound having the structure: 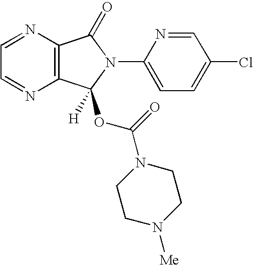
or a pharmaceutically acceptable salt, solvate, hydrate, polymorph, or prodrug thereof,
wherein the unit dosage form has an AUC of at least 120 percent greater than a reference eszopiclone formulation.

3. A unit dosage form comprising a compound having the structure: 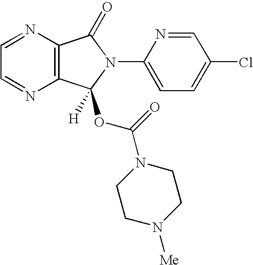
or a pharmaceutically acceptable salt, solvate, hydrate, polymorph, or prodrug thereof,
said compound being present in said dosage form in an amount or in a sustained release component effective to achieve a maximum plasma concentration (Cₘₐₓ) insufficient to induce moderate sedation in a subject to which said unit dosage form is administered.

4. The unit dosage form of any one of claims 1 through 3 comprising up to 0.8 mg of the compound.

5. The unit dosage form of any one of claims 1 through 3 comprising up to 0.7 mg of the compound.

6. The unit dosage form of any one of claims 1 through 3 comprising up to 0.6 mg of the compound.

7. The unit dosage form of any one of claims 1 through 3 comprising up to 0.5 mg of the compound.

8. The unit dosage form of any one of claims 1 through 3 comprising up to 0.4 mg of the compound.

9. The unit dosage form of claims 2 or 3 comprising up to 3 mg of the compound.

10. The unit dosage form of claims 2 or 3 comprising up to 2 mg of the compound.

11. The unit dosage form of claims 2 or 3 comprising up to 1.5 mg of the compound.

12. The unit dosage form of claims 2 or 3 comprising up to 1 mg of the compound.

13. The unit dosage form of any one of claims 1 through 3 wherein the unit dosage form has an AUC of at least 150 percent greater than a reference eszopiclone formulation.

14. The unit dosage form of any one of claims 1 through 3 wherein the unit dosage form has an AUC of at least 200 percent greater than a reference eszopiclone formulation.

15. The unit dosage form of any one of claims 1 through 3 wherein the unit dosage form has an AUC of at least 400 percent greater than a reference eszopiclone formulation.

16. The unit dosage form of any one of claims 1 through 3 wherein the unit dosage form does not exceed the Cₘₐₓ of a reference eszopiclone formulation by more than about 150 percent.

17. The unit dosage form of any one of claims 1 through 3 wherein the unit dosage form does not exceed the Cₘₐₓ of a reference eszopiclone formulation by more than about 200 percent.

18. The unit dosage form of any one of claims 1 through 3 wherein the unit dosage form does not exceed the Cₘₐₓ of a reference eszopiclone formulation by more than about 300 percent.

19. The unit dosage form of claim 2 wherein the reference eszopiclone formulation is an instantaneous release formulation that contains one mg eszopiclone and provides in a 70 kg subject a Cₘₐₓ of eszopiclone of 10 ng/ml, a Tₘₐₓ of 1.5 hours and a drug half-life of 6 hours.

20. The unit dosage form of claim 2 wherein the reference eszopiclone formulation is an instantaneous release formulation and the unit dosage form and the reference eszopiclone formulation each contain the same amount by weight of eszopiclone.

21. The unit dosage form of any one of claims 1 through 3 further comprising a member selected from: (a) an adjuvant; (b) an anti-oxidant; (c) a buffer; (d) a carrier; (e) a colorant; (f) a diluent; (g) a disintegrant; (h) an excipient; (i) a filler; (j) a flavorant; (k) a gelling agent; (l) a lubricant; (m) a neutralizing agent; (n) a preservative; and (o) any combination of any of the foregoing.

22. The unit dosage form of any one of claims 1 through 3 comprising 0.1% or more by weight of said compound.

23. The unit dosage form of any one of claims 1 through 3 comprising 1% or more by weight of said compound.

24. The unit dosage form of any one of claims 1 through 3 comprising 2% or more by weight of said compound.

25. The unit dosage form of any one of claims 1 through 3 comprising 3% or more by weight of said compound.

26. The unit dosage form of any one of claims 1 through 3 wherein said compound present in said unit dosage form is present in at least about 99.5% enantiomeric excess.

27. The unit dosage form of any one of claims 1 through 3 wherein said compound present in said unit dosage form is present in at least about 99.9% enantiomeric excess.

28. The unit dosage form of any one of claims 1 through 3 wherein said composition is essentially free of an antipodal enantiomer of said compound.

29. The unit dosage form of any one of claims 1 through 3 comprising said compound in an amount sufficient to achieve a maximum plasma concentration (Cₘₐₓ) of from about 0.1 ng/mL to about 25 ng/mL of said compound.

30. The unit dosage form of any one of claims 1 through 3 comprising said compound in an amount sufficient to achieve a maximum plasma concentration (Cₘₐₓ) in said subject of from about 0.5 ng/mL to about 20 ng/mL of said compound.

31. The unit dosage form of any one of claims 1 through 3 comprising said compound in an amount sufficient to achieve a maximum plasma concentration (Cₘₐₓ) in said subject of from about 1 ng/mL to about 10 ng/mL of said compound.

32. The unit dosage form of any one of claims 1 through 3 comprising said compound in an amount sufficient to achieve a maximum plasma concentration (Cₘₐₓ) in said subject of from about 2 ng/mL to about 8 ng/mL of said compound.

33. The unit dosage form of any one of claims 1 through 3 comprising said compound in an amount sufficient to achieve a maximum plasma concentration (Cₘₐₓ) in said subject of from about 3 ng/mL to about 5 ng/mL of said compound.

34. The unit dosage form of any one of claims 1 through 3 comprising said compound in an amount sufficient to achieve a maximum plasma concentration (Cₘₐₓ) in said subject of not more than about 20 ng/mL of said compound.

35. The unit dosage form of any one of claims 1 through 3 comprising said compound in an amount sufficient to achieve a maximum plasma concentration (Cₘₐₓ) in said subject of not more than about 8 ng/mL of said compound.

36. The unit dosage form of any one of claims 1 through 3 comprising said compound in an amount sufficient to provide to said subject a dose of less than about 4 mg/70 kg drug/patient weight.

37. The unit dosage form of any one of claims 1 through 3 comprising said compound in an amount sufficient to provide to said subject a dose of from about 0.25 mg/70 kg to about 0.9 mg/70 kg drug/patient weight.

38. The unit dosage form of any one of claims 1 through 3 comprising said compound in an amount sufficient to provide to said subject a dose of from about 0.5 mg/70 kg to about 0.9 mg/70 kg drug/patient weight.

39. The unit dosage form of any one of claims 1 through 3 comprising said compound in an amount sufficient to provide to said subject a dose of less than about 1 mg/70 kg drug/patient weight.

40. The unit dosage form of any one of claims 1 through 3 selected from the group consisting of tablets, pills, capsules, boluses, powders, granules, sterile parenteral solutions, sterile parenteral suspensions, elixirs, tinctures, metered aerosol, liquid sprays, drops, ampoules, autoinjector devices, suppositories, transdermal patches, and a lyophilized composition.

41. The unit dosage form of any one of claims 1 through 3 wherein said compound is present in an amount sufficient to induce anxiolysis in said subject.

42. The unit dosage form of any one of claims 1 through 3 wherein the dosage form is a sustained release formulation.

43. The unit dosage form of any one of claims 1 through 3 which comprises a sustained release component comprising said compound and an instantaneous release component comprising said compound.

44. The unit dosage form of claim 43 wherein said sustained release component and said instantaneous release component are each in a separate compartment of said unit dosage form.

45. The unit dosage form of claim 43 wherein said separate compartments are isolated compartments separated by a physical barrier.

46. The unit dosage form of claim 43 wherein said separate compartments are particles of said compound of a first size and a second size, respectively, wherein said particles of said first size and said particles of said second size release said compound into said plasma at a first rate and a second rate, respectively, wherein said first rate and said second rate are different.

47. The unit dosage form of claim 42 wherein said sustained release component comprises said compound within a polymeric matrix.

48. The unit dosage form of any one of claims 1 through 3 wherein said dosage form comprises an amount of said compound sufficient to provide a plasma concentration of said compound, in a subject to which said composition is administered, which is anxiolytic for a period of at least about 6 hours, without being moderately sedative during said period.

49. The unit dosage form of any one of claims 1 through 3 wherein said dosage form comprises an amount of said compound sufficient to provide a plasma concentration of said compound, in a subject to which said composition is administered, which is anxiolytic for a period of at least about 8 hours, without being moderately sedative during said period.

50. The unit dosage form of any one of claims 1 through 3 wherein said dosage form comprises an amount of said compound sufficient to provide a plasma concentration of said compound, in a subject to which said composition is administered, which is anxiolytic for a period of at least about 10 hours, without being moderately sedative during said period.

51. The unit dosage form of any one of claims 1 through 3 wherein said dosage form comprises an amount of said compound sufficient to provide a plasma concentration of said compound, in a subject to which said composition is administered, which is anxiolytic for a period of at least about 12 hours, without being moderately sedative during said period.

52. The unit dosage form of any one of claims 1 through 3 wherein at least about 80% of said compound is present in particles of a size less than or equal to about 50 μm.

53-60. (canceled)

61. The unit dosage form of any one of claims 1 through 3 comprising the racemate zopiclone in place of eszopiclone.

62. A method of inducing anxiolysis in a subject in need thereof, said method comprising, administering to said subject a unit dosage form comprising an amount of a compound having the structure: 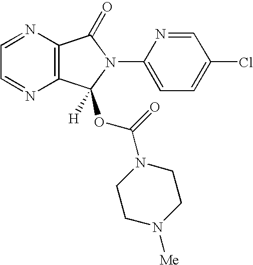
sufficient to induce anxiolysis.

63. (canceled)

64. The method of claim 62 wherein the compound is administered at least two hours outside of the subject's normal sleep cycle.

65. The method of claim 62 wherein the compound is administered at least four hours outside of the subject's normal sleep cycle.

66. The method of claim 62 wherein the compound is administered between 6:00 am and 6:00 pm.

67. The method of claim 62 wherein the compound is administered between 6:00 am and 4:00 pm.

68. The method of claim 62 comprising the racemate zopiclone in place of eszopiclone.

69-72. (canceled)

73. A method of inducing anxiolysis in a subject in need thereof, said method comprising, administering to said subject a unit dosage form comprising up to 0.9 mg of a compound having the structure: 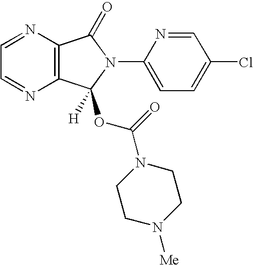
or a pharmaceutically acceptable salt, solvate, hydrate, polymorph, or prodrug thereof.

74. A method of inducing anxiolysis in a subject in need thereof, said method comprising, administering to said subject a modified dosage form comprising a compound having the structure: 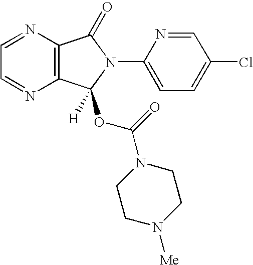
or a pharmaceutically acceptable salt, solvate, hydrate, polymorph, or prodrug thereof,
wherein the dosage form has an AUC of at least 120 percent greater than a reference eszopiclone formulation.

75. A method of inducing anxiolysis in a subject in need thereof, said method comprising, administering to said subject a modified dosage form comprising a compound having the structure: 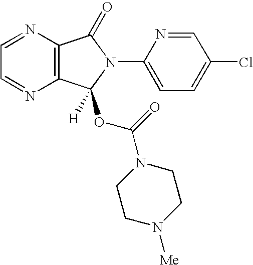
or a pharmaceutically acceptable salt, solvate, hydrate, polymorph, or prodrug thereof,
said compound being present in said dosage form in an amount or in a sustained release component effective to achieve a maximum plasma concentration (Cₘₐₓ) insufficient to induce moderate sedation in a subject to which said unit dosage form is administered.

76. A method of inducing anxiolysis in a subject in need thereof, said method comprising, administering to said subject a unit dosage form comprising a compound having the structure: 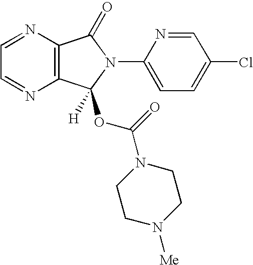
said unit dosage form comprising said compound in a sustained release component of said formulation in an amount effective to achieve a maximum plasma concentration (Cₘₐₓ) insufficient to induce moderate sedation in a subject to which said unit dosage form is administered for a period of at least 6 hours.

77. A method of inducing anxiolysis in a subject in need thereof, said method comprising, administering to said subject a compound having the structure: 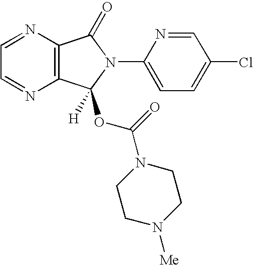
such that a plasma concentration of said compound is sufficient to induce anxiolysis in said subject for at least about 6 hours and a maximum plasma concentration (Cₘₐₓ) is insufficient to moderately sedate said subject for greater than 1 hour.

78-81. (canceled)

82. The method of claim 62 further comprising identifying and selecting a subject in need of treatment of anxiety and administering the compound to the identified and selected subject.
